(19) United States
(12) Patent Application Publication
(10) Pub. No.: US 2006/0251672 A1
(43) Pub. Date: Nov. 9, 2006

(54) Composition and methods of treating allergies with reishi extract

(76) Inventors: De-Cheng Zhang, Shanghai (CN); Can-Jun Li, Shanghai (CN); Jia-shi Zhu, San Diego, CA (US); Howard Sun, Draper, UT (US)

Correspondence Address: THORPE NORTH & WESTERN, LLP., 8180 SOUTH 700 EAST, SUITE 200, SANDY, UT, 84070, US

(21) Appl. No.: 11/122,265

(22) Filed: May 3, 2005

(51) Int. Cl. A61K 36/06

(52) U.S. Cl. 424/195.15

(57) ABSTRACT

Reishi extract compositions and methods for preparation and use thereof are disclosed and described. In various aspects, methods for inhibiting IL-5 secretion, and preventing and/or treating allergies are described, which include administering therapeutically effective amounts of compositions containing reishi extracts to subjects.

### FIELD OF THE INVENTION

The present invention relates to reishi extract compositions and uses thereof. Accordingly, the present invention involves the areas of botany, nutritional and health sciences, as well as medicine and pharmaceutical/nutraceutical sciences.

### BACKGROUND OF THE INVENTION

Allergies are a nuisance to many people, and can sometimes cause serious heath risks. Allergic diseases can elicit inflammatory responses or reactions ranging from mild itchy skin and watery eyes to life-threatening anaphylactic shock. It is common for individuals suffering from allergies to experience decreased personal performance, which can directly result in a poor physical and/or emotional condition. Accordingly, the pursuit of allergy related therapies is of great interest to the health care industry.

An allergic reaction occurs when an allergic individual exhibits an abnormally high sensitivity to a substance or compound, resulting in the stimulation of the individual's immune system to react against an allergen. Allergic reactions can be classified into four general types including, Type I, Type II, Type III, and Type IV as in Taber's Cyclopedic Medical Dictionary, 18th Ed., F. A. Davis Company, 67-68 (1997), which is incorporated herein by reference.

Typically, the immune system is not stimulated the first time the allergen is presented. During the first exposure, it is thought that the allergen comes into contact with antigen expressing cells (e.g., macrophages or dendritic cells), which degrade the allergen into fragments to be presented to T cells. Accordingly, the T cells are activated and induced into cytokine production and secretion as a Th-2 response. These Th-2 cytokines are secreted factors that induce terminal differentiation of late-developing B-cells into immunoglobulin secreting cells. Cytokine secretion can increase maturation of B cells, which produce IgE antibodies specific to antigens expressed on the allergen. Upon a subsequent exposure, the IgE class antibodies are available to bind to the antigens on the allergen.

Recently, there has been considerable progress in understanding the basis by which cytokines released from T cells contribute to allergic diseases. A subset of T cells (Th-2 cells) produce cytokines, such as interleukin-5 (IL-5), during a Th-2 response. IL-5 is a 15 kD protein of 134 amino acids, and is known to be involved in T-cell replacing factor (TRF), eosinophil differentiation factor, and B-cell differentiation factor. The cytokine IL-5 has been implicated in the pathogenesis of allergen induced reactions, where increased IL-5 secretion can stimulate eosinophilic inflammation. Accordingly, the secretion of the cytokine IL-5 can increase the persistence of an allergic inflammation to a late stage allergic reaction. Thus, IL-5 may be a factor in governing eosinophilic inflammation of airway tissue, and may play a role in the pulmonary eosinophilia associated with allergic reactions.

When eosinophils are stimulated by IL-5, they can destroy tissues and cells, which leads to further complications. In seasonal allergic rhinitis (SAR), which is commonly experienced by individuals with asthmatic conditions, IL-5 has been shown to enhance nasal allergic inflammation that enhances eosinophil production causing airway inflammation. The involvement of IL-5 in allergic pathways was explored in the article, A single nasal allergen challenge increases induced sputum inflammatory markers in non-asthmatic subjects with seasonal allergic rhinitis: correlation with plasma interleukin-5; Beeh K M, Beier J, Kornmann O, Meier C, Taeumer T, Buhl R. Clin Exp Allergy, April;33(4):475-82 (2003), incorporated herein by reference. Also, IL-5 has been shown to be involved in eosinophil growth, maturation, and accumulation upon an allergen challenge to individuals suffering from an asthmatic condition as shown in the article, Allergen-induced increases in bone marrow T lymphocytes and interleukin-5 expression in subjects with asthma; Wood L J, Sehmi R, Dorman S, Hamid Q, Tulic M K, Watson R M, Foley R, Wasi P, Denburg J A, Gauvreau G, O'Byrne P M. Am J Respir Crit Care Med (2002) September 15;166(6):883-9, incorporated herein by reference.

In short, IL-5 plays a role in the process responsible for controlling the eosinophilic inflammation characteristics of an asthmatic and/or allergic reaction. Thus, the down-regulation of IL-5 secretion or reduction of a Th-2 response has been a goal for the treatment of allergic diseases such as asthma, allergic rhinitis, allergic conjunctivitis and atopic eczema among others.

### SUMMARY OF THE INVENTION

Accordingly, the present invention encompasses compositions and methods for reducing IL-5 secretion in a subject. In one aspect, the present invention provides formulations that may comprise, consist essentially of, or consist of a therapeutically effective amount of a reishi extract. Such extracts have been found to inhibit IL-5 secretion when administered to a subject. In another aspect, additional active ingredients may be combined with the reishi extract in the formulations. Such formulations may be suitably prepared into a number of dosage forms for administration to a subject by combination with yet additional ingredients such as an inert carrier. Examples of suitable dosage forms include without limitation oral, parenteral, transdermal and/or transmucosal dosage forms.

Another aspect of the present invention provides methods of inhibiting IL-5 secretion in a subject. Such methods may be employed by providing a composition containing a therapeutically effective amount of reishi extract, and administering the composition to the subject. Specific dosage regimens that are sufficient to achieve and/or maintain the inhibition of IL-5 secretion in the subject over a defined period of time, may be determined by those of ordinary skill in the art using their specific knowledge of the subject.

Another aspect of the present invention provides methods of treating or preventing an allergic reaction in a subject. Such methods may include providing a composition containing a therapeutically effective amount of reishi extract, and administering the composition to the subject in an amount sufficient to inhibit the secretion of IL-5 in the subject when exposed to an allergen. Again, specific dosage regimens that are sufficient to treat or prevent an allergic reaction in a subject over a defined period of time, may be determined by those of ordinary skill in the art, using their specific knowledge of the subject.

Another aspect of the present invention provides methods of making a reishi composition. In one aspect, such methods may include the steps of: 1) providing at least a portion of a reishi mushroom, 2) treating the portion of reishi mushroom with a 65-95% aqueous alcohol solution to obtain a crude triterpene extract, 3) concentrating the crude triterpene extract to obtain at least one triterpene, 4) treating the portion of a reishi mushroom with a solvent to obtain a crude polysaccharide extract, 5) treating the crude polysaccharide extract with ethanol to obtain at least one polysaccharide, and 6) forming a combined composition by combining at least one triterpene and at least one polysaccharide. Notably, an aspect of the invention allows for the same portion of a reishi mushroom to be used for obtaining the crude triterpene extract as well as for obtaining the crude polysaccharide extract. Also, another aspect allows for different portions of a reishi mushroom to be used for obtaining the crude triterpene extract and polysaccharide extract. Further, an aspect of the invention allows for providing additional steps in both preparing the portion of reishi mushroom, and purifying the extracts therefrom.

Thus, the more important features of the invention have been broadly outlined. The above-recited aspects of the invention may be better understood from a consideration of the following detailed description. Also, other features of the present invention will become clearer from the detailed description that follows, taken with the claims, or may be learned by the practice of the invention.

### DETAILED DESCRIPTION OF THE INVENTION

Before the compositions and accompanying methods of the present invention are disclosed and described, it is to be understood that this invention is not limited to the particular process steps and materials disclosed herein, but is extended to equivalents thereof, as would be recognized by those of ordinary skill in the relevant arts. Also, it should be understood that the terminology employed herein is only used for the purpose of describing particular embodiments of the invention and is not intended to be limiting in any manner.

Definitions

In describing and claiming the present invention, the following terminology will be used in accordance with the definitions set forth below.

The singular forms “a,” “an,” and, “the” include plural referents unless the context clearly dictates otherwise. Thus, for example, reference to “a carrier” includes reference to one or more of such carriers, and reference to “an active agent” includes reference to one or more of such active agents.

As used herein, the terms “formulation” and “composition” may be used interchangeably. The terms “drug,” “active agent,” “bioactive agent,” “pharmaceutically active agent,” “nutraceutical active agent,” “pharmaceutical,” and “nutraceutical,” are also used interchangeably to refer to an agent or substance that has measurable specified or selected physiologic activity when administered to a subject in an effective amount. These terms of art are well-known in the pharmaceutical, nutraceutical, and medicinal arts.

As used herein, the terms “administration,” and “administering” refer to the manner in which a formulation or composition is introduced into the body of a subject. Administration can be accomplished by various art-known routes such as oral, parenteral, transdermal, inhalation, implantation, etc. Thus, oral administration can be achieved by swallowing, chewing, and/or sucking of an oral dosage form comprising an active agent. Parenteral administration can be achieved by injecting a drug composition intravenously, intra-arterially, intramuscularly, intrathecally, or subcutaneously, etc. Transdermal administration can be accomplished by applying, affixing, pasting, rolling, attaching, pouring, pressing, rubbing, etc., of a transdermal preparation onto a skin surface. These and additional methods of administration are well-known in the art.

The terms “effective amount” and “sufficient amount” may be used interchangeably and refer to an amount of an ingredient which, when included in a composition, is sufficient to achieve an intended compositional or physiological effect. Thus, a “therapeutically effective amount” refers to a non-toxic, but sufficient amount of an active agent, to achieve therapeutic results in treating a condition for which the active agent is known to be effective. Various biological factors may affect the ability of a substance to perform its intended task. Therefore, an “effective amount” or a “therapeutically effective amount” may be dependent on such biological factors. Further, while the achievement of therapeutic effects may be measured by a physician or other qualified medical personnel using evaluations known in the art, it is recognized that individual variations and responses to treatments may make the achievement of therapeutic effects a subjective decision. Accordingly, the determination of an effective amount is well within the ordinary skill in the art of pharmaceutical, nutraceutical, herbaceutical, and health sciences. See, Meiner and Tonascia, “Clinical Trials: Design, Conduct, and Analysis,” Monographs in Epidemiology and Biostatistics, Vol. 8 (1986), incorporated herein by reference.

As used herein, the terms “carrier” “pharmaceutically acceptable carrier” or “inert carrier” refer to a carrier vehicle with which a bioactive agent, such as a reishi extract, may be combined to achieve a specific dosage form. As a generally principle, carriers must not react with the bioactive agent in a manner which substantially degrades or otherwise adversely affects the bioactive agent. Other “inactive” ingredients may also be used in creating reishi extract formulations having specifically desired properties or dosage forms, and will be readily recognized by those of ordinary skill in the art.

As used herein, the term “subject” refers to a mammal that may benefit from the administration of an allergy and/or asthma treatment, or IL-5 secretion reducing composition or method as recited herein. Most often, the subject will be a human.

The term “reishi,” refers to the mushroom species Ganoderma lucidum, including all strains and hybrids thereof, grown anywhere in the world. Reishi is also known by various common names such as ling zhi and young zhi.

The term “extract,” when used in connection with a reishi mushroom, refers to one or more active agents, or a composition containing such, that is obtained from the mushroom, or a portion thereof. The extract may be derived from any aspect of a mushroom including without limitation, the cap, gills, spines, pores, spores, fruiting bodies, veil, stem, mycelium, hyphae, rhizomorph, pileus, and any other aspect of a mushroom. As will be recognized by those of ordinary skill in the art, extracts may be either crude or refined to a selected degree in order to isolate certain active agents. A number of extraction processes can be employed to produce compositions of various types, and will be recognized by those of ordinary skill in the art. Also, an extract can be in any physical phase, consistency, and/or form, and is not limited to being a solid, a dry powder, a liquid, a paste, or other physical characteristic designation.

As used herein, the terms “inhibit,” “inhibiting,” or any other derivative thereof refers to the process of holding back, suppressing or restraining so as to block, prevent, limit, or decrease a rate of action or function. The use of the term is not to be misconstrued to be only of absolute prevention, but can be a referent to any minute incremental step of limiting or reducing a function through the full and absolute prevention of the function.

The term “secretion,” or any derivative thereof refers to the physiological processes of substance generation and/or separation from either the cells or bodily fluids of a subject. Thus, the secretion of IL-5 refers to the entire physiological process required for releasing IL-5 from a cell into an extra-cellular space, which includes starting with the intra-cellular machinery building the cytokine through the release of IL-5 from the cell. Further, use of the term “secretion” in conjunction with the term “inhibiting” may refer to the entire physiological process within a subject of recognizing a sensitized stimulus, and having some point within the cascading sequence of events interrupted to prevent an increase of a particular substance either in the cell or in the subject outside of the substance-producing cell, i.e., preventing an increase in IL-5. Accordingly, Taber's Cyclopedic Medical Dictionary, 18th Ed., F. A. Davis Company, 1732-1733 (1997), is incorporated herein by reference.

As used herein, the term “cracked,” or any other derivative thereof refers to a process of chemically and/or physically breaking or snapping something apart, which can include breaking without the complete separation of parts. For example, reishi spores are minute brown egg-shaped reproductive cells that are released by the mushroom at maturity. The spores are protected by an extremely hard shell, which prevents certain internal contents in the spore from being available outside the spore. Consequently, when the spore is “cracked” the hard shell is at least fissured so that the internal contents are then available and can be removed from the spore. Thus use of the term “cracked” in association with the term “spore” identifies the spore is in a physical state that allows the internal contents as well as the external components to be available.

As used herein, the terms “asthmatic condition,” “asthma,” or any derivative thereof refers to a subject with a condition of “increased responsiveness of the tracheobronchial tree to various stimuli, which results in paroxysmal constriction of the bronchial airways.” See, Taber's Cyclopedic Medical Dictionary, 18th Ed., F. A. Davis Company, 164-165 (1997), which is incorporated herein by reference.

The term “allergy” or any derivative thereof refers to an abnormal response to a substance that normally does not cause a reaction in a subject. Usually, sensitization to a substance is required from an initial exposure, where a subsequent contact with the substance can result in a broad range of reactions, i.e., an allergic reaction. An allergy may be represented by any of four types of reactions, which are designated by Type I, Type II, Type III, and Type IV as defined in, Taber's Cyclopedic Medical Dictionary, 18th Ed., F. A. Davis Company, 67-68 (1997), which is incorporated herein by reference.

The phrase “allergic reaction” or any derivative thereof refers to the physiological response in a subject to an allergen when the subject is allergic to the allergen. The symptoms of an allergic reaction can range from a mild irritation to severe health complications, which depend on an individual's tolerance to the allergen. Accordingly, the symptoms of an allergic reaction include without limitation, watery and/or red eyes, itching, sneezing, wheezing, breathing difficulties, a runny or bleeding nose, hives, swelling of the throat, swelling of the lips, swelling of the tongue, swelling around the eyes, bloating, cramps, abnormal stools, aching, anorexia, bloating, compulsive eating, diarrhea, drowsiness, edema, erythema, fatigue, fever, flatulence, headache, hypotension, indigestion, irritability, itching, malaise, nasal congestion, nausea, ocular pruritus, pruritus, rash, sleep disturbances, sore throat, stiffness, sweating, swelling, vomiting, and weight loss. The most severe form of an allergic reaction is anaphylactic shock, which can be a medical emergency that results in the loss of consciousness or even death.

The term “allergen” or any derivative thereof refers to any substance, particulate, or antigen that may cause physical manifestations associated with an allergy. Examples of some typical allergens include without limitation, those that are inhaled such as dusts, pollens, fungi, smoke, perfumes, airborne particulates of plastics etc.; foods such as wheat, eggs, milk, chocolate, strawberries, etc.; drugs such as aspirin, antibiotics, serums, etc.; infectious agents such as bacteria, viruses, fungi animal parasites, etc.; and contactants such chemicals, animals, plants, metals, etc. See, Taber's Cyclopedic Medical Dictionary, 18th Ed., F. A. Davis Company, 67 (1997), which is incorporated herein by reference. These examples of allergens are not to be construed as limiting and only represent an aspect of the entire spectrum of possible allergens.

The term “aeroallergen” or any derivative thereof refers to an airborne allergen. That is, when an allergen is sufficiently small as to enable the allergen to be present in the air, the allergen is an “aeroallergen.” Aeroallergens include without limitation dust, molds, particulates, chemicals, and latex particles, where the subject's exposure may be from either inhalation or contact with the dermis.

When an allergen is from exposure to an animal, the subject comes into contact with the allergen. Proteins and/or particulates of or from the dander, urine, hair, or saliva of warm-blooded animals (e.g., cats, dogs, mice, rats, gerbils, birds, etc.) have been reported to sensitize individuals and can cause allergic reactions or asthmatic conditions in individuals sensitive to the animal allergens. The primary cat allergen, felis domesticus allergen I, is a glycoprotein found in the sebaceous glands of the cat's hair roots, in their sublingual salivary glands, and in the urine of male cats. These animal allergens are typically microscopic particles, which easily can become and remain airborne for long periods of time. Direct contact with the animal presenting these allergen particles can cause the allergen to come into contact with the eyes, nose, or bronchial tubes, and provoke an allergic reaction. Animal allergens are found mostly in homes where pets are present, and can also be found in places where pets have never been present, such as schools, workplaces, and other public spaces. An animal allergen can be brought to these places on the clothing of pet owners or others in contact with the animal, and can cause an allergic reaction by indirect contact.

Concentrations, amounts, solubilities, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a concentration range of 0.01 to 6 mg/kg should be interpreted to include not only the explicitly recited concentration limits of 0.01 mg/kg and 6 mg/kg, but also to include individual concentrations such as 0.3 mg/kg, 0.6 mg/kg, 2.0 mg/kg, 2.3 mg/kg, 3.7 mg/kg, 5.4 mg/kg, and sub-ranges such as 0.2-2.3 mg/kg, 1.3-3.9 mg/kg, 2.9-5.1 mg/kg, etc. This interpretation should apply regardless of the breadth of the range or the characteristic being described, and should apply to ranges having both upper and lower numerical values, as well as open-ended ranges reciting only one numerical value.

The Invention

Applicants have discovered that a formulation containing at least a portion of a reishi mushroom reduces IL-5 secretion in a subject when administered thereto. Also, the Applicants have discovered methods of making a reishi containing formulation, methods of administering the formulation to inhibit IL-5 secretion, and methods of administering the formulation to treat or prevent asthma and/or an allergic reaction. As such, the present invention extends not only to the composition of the formulation, but also to the process of making the formulation, and methods of using the formulation as indicated.

In accordance with the present invention, a composition that comprises, consists essentially of, or consists of a reishi extract may be administered to a subject in a therapeutically effective amount to inhibit IL-5 secretion. The reishi compositions of the present invention may be formulated into a variety of suitable dosage forms for the administration thereof. In one aspect, the reishi extract may be combined with carriers and/or other inactive ingredients in order to create a specific dosage form. Many different dosage forms are well known to those of ordinary skill in the art and may be used for the administration of the reishi extract. In one aspect, such a composition may consist essentially of a therapeutically effective amount of a reishi extract.

In another aspect, the composition may also include other active agents in addition to the reishi extract, which are included to provide an intended effect, or a specifically desired result. Both natural and synthetically produced active agents may be included. Those of ordinary skill in the art will be able to select from a wide range of specific ingredients in order to provide a desired therapeutic effect. In one aspect, the additional active ingredient may be a natural ingredient, such as an herbal or botanical extract. In another aspect, the additional active ingredient may be synthetically produced.

In addition to the aforementioned composition, the reishi extract utilized in the present invention may be derived from any part of the reishi mushroom, and may take a variety of physical forms. However, in one aspect the extract may be an oil. In another aspect, the extract may be a solid. In yet another aspect, the extract may be a water- and/or organic-soluble infusion extract. In a further aspect, the extract may be a dry, or lyophilized powder. In an additional aspect, the extract may be an emulsion. Moreover, the extract may be obtained from various portions of the reishi mushroom. In one aspect, the extract may be obtained from the fruit. In another aspect, the extract may be obtained from the spores.

The reishi formulation may be provided as an oral dosage form. A variety of oral dosage forms are well known to those of ordinary skill in the art, and specific formulation ingredients may be selected in order to provide a specific result. Examples of oral dosage forms include without limitation, oral dosage forms such as tablets, capsules, gel capsules, liquids, syrups, elixirs, and suspensions. Additionally, oral dosage forms encompass food preparations, such as bars and beverages.

The reishi formulation may be provided as a transdermal or parenteral dosage form. A number of specific transdermal and parenteral dosage forms are well known to those of ordinary skill in the art. Examples of transdermal dosage forms include without limitation lotions, gels, creams, pastes, ointments, transmucosal tablets, adhesive devices, adhesive matrix-type transdermal patches, liquid reservoir transdermal patches, etc. Also, parenteral dosage forms may be formulated to provide efficient delivery of reishi extract to a subject.

With the aforementioned compositions in mind, the reishi extract may comprise at least one triterpene compound and/or at least one polysaccharide compound. In another aspect, the reishi extract may comprise from about 5 to 10% triterpene compounds and/or from about 12 to 18% polysaccharide compounds. In still another aspect, the reishi extract may comprise, consist essentially of, or consist of about 80-99% reishi fruit extract and of about 1-20% cracked reishi spore.

In regard to the compositions containing reishi discussed herein, a composition may include a cracked reishi spore extract. Also, the composition may include a reishi fruit extract. In another aspect, the composition may include a reishi fruit extract and a cracked reishi spore extract. Further, the composition may comprise from about 247.5 mg to 990 mg of reishi fruit extract and from about 2.5 to 10 mg of cracked reishi spore extract. Furthermore, the composition may comprise about 495 mg of reishi fruit extract and about 5 mg of cracked reishi spore extract. As used herein, a reishi fruit extract can be from any aspect of a reishi mushroom excluding the reishi spore, and vice versa.

As discussed above, the present invention additionally encompasses methods for using the reishi formulations disclosed herein. In one aspect, the present invention provides a method for inhibiting IL-5 secretion in a subject by providing a composition containing a therapeutically effective amount of reishi extract, and administered the composition to a subject. In another aspect, the inhibition of IL-5 secretion treats or prevents an asthmatic condition.

Similarly, the present invention provides a method of using the reishi formulations disclosed herein for preventing an allergic reaction in a subject. In yet another aspect, the present invention provides a method for treating an allergic reaction in a subject. Such methods may include the steps of providing a composition containing a therapeutically effective amount of reishi extract, and administering the composition to the subject in an amount sufficient to inhibit the secretion of IL-5 in the subject when the subject is exposed to an allergen.

With the above methods of using a reishi composition in mind, an additional aspect of the invention provides a method of preventing or treating an allergic reaction, where the allergic reaction can be asthma, allergic rhinitis, allergic conjunctivitis, or atopic exzema. Another aspect of the invention provides a method for preventing or treating a Type I, Type II, Type III, or Type IV allergic reaction.

In addition, the present invention provides a method of preventing or treating an allergic reaction to an allergen, where the allergen is ingested, from dermal contact, or inhaled. Additionally, the allergen may be from ingestion of a peanut, exposure to a pollen particle, exposure to a mold particle, or exposure to a dust particle. Further, the allergen may be from exposure to an animal, where the allergen can be from dander, hair, urine, feces, and protein substances. Furthermore, the allergen may be an aeroallergen.

In regard of the reishi compositions and uses thereof, another aspect of the present invention provides for using a reishi composition that may be tailored for specific dosage regimens. The specific dosage form and amount of reishi contained therein may be designed to attain a desired concentration of reishi extract active ingredients in a subject. Thus, those of ordinary skill in the art will be able to determine proper dosage amounts for specific individuals through routine monitoring and adjustment of the physiological effects of a given dosage. However, as a general matter, a reishi composition may be administered in an amount and frequency sufficient to provide a daily dosage of about 495 mg to 1980 mg of reishi fruit extract and of about 5 mg to 20 mg of cracked reishi spore extract. In another aspect, the reishi composition may be administered in an amount and frequency sufficient to provide a daily dosage of about 990 mg of reishi fruit extract and of about 10 mg of cracked reishi spore extract. In a further aspect, the reishi composition may be administered in an amount and frequency sufficient to provide a daily dosage of from about 1 to 2 grams of reishi extract.

With the aforementioned compositions and methods of use in mind, the method of making a reishi composition of the present invention may include the following general scheme. After any aspect of a reishi mushroom material has been obtained, it may be partitioned into portions. Some portions of reishi mushroom can be treated with an 80% aqueous alcohol solution to obtain a crude triterpene extract. The crude triterpene extract can then be concentrated to obtain at least one triterpene. Also, some portions of reishi mushroom can be treated with a solvent to obtain a crude polysaccharide extract. In one aspect of the present invention, the solvent can be acidic. In another aspect, the solvent can be alkaline. In yet another aspect, the solvent can be neutral. The crude polysaccharide extract can then be treated with ethanol to concentrate the polysaccharides and obtain at least one polysaccharide. In another aspect, treating the crude polysaccharide extract with ethanol can precipitate at least one polysaccharide. Further, at least one triterpene and at least one polysaccharide can be combined to form a combined composition, which may be referred to as a reishi composition and/or a reishi extract.

Also, the method of making a reishi composition can include additional general steps. Accordingly, a portion of a reishi mushroom including a cell wall can additionally be provided. In addition, at least one polysaccharide can be detached from the cell wall to obtain a crude polysaccharide extract. In a further aspect of the invention, the portion of reishi mushroom comprises, consists essentially of, or consists of a cracked reishi spore. In another aspect, the portion of reishi mushroom comprises, consists essentially of, or consists of a reishi fruit. In still a further aspect, the portion of reishi mushroom consists essentially of a cracked reishi spore and a reishi fruit.

In another aspect of the present invention, the reishi mushroom may be cultivated by a solid wood log cultivation process. Artificial cultivation is the process of affirmatively taking action to grow a reishi mushroom rather than rely on Mother Nature's natural slow growth cycle. Artificial cultivation may be by a sawdust method or a solid wood log method, where the solid wood log method can result in superior reishi. The solid wood log cultivation process can be used during outdoor or indoor cultivation and makes use of natural bed wood logs. The wood logs can include without limitation, a white oak (quercus motiogolica), an oak (quercus acutissima), a Japanese chestnut (castanea sp), and Japanese apricot (prunus mume) for mass-cultivation of high quality reishi mushrooms, but other types of wood logs may be used. Also, heat-treated wood logs can be used for culture, which can additionally be used for unclosed seeding. The wood logs may be cut into pieces of about 20 centimeters length and about 25 centimeters in diameter, but there is no strict limitation on the size or dimensions of wood logs. Spawning may be conducted by making about 6-10 holes of about 2 centimeters in diameter and about 5 centimeters deep, and placing spawn therein, where there is not strict limitation on numbers or dimensions. Sealing the spawned holes can prevent the spawn from being dried or contaminated with germs, which can be done with melted wax or any equivalent. For propagating the spawn all over the wood logs, the spawned wood logs are kept for 6 months in a cleaned culture room controlled at about 25-30° C., and about 70-90% humidity. After propagation of the spawn, the spawned wood logs can be buried into enriched soil in a heated greenhouse. Inside the greenhouse irrigation can be provided, and the air can be controlled at about 25-30° C. and about 70-90% humidity. Also, the greenhouse can be blocked from direct rays and can be well ventilated for the reishi mushrooms to grow. In the final stage of growth, when the spores are on the down side of the pileus, the reishi mushrooms need to be watered for about a week, then again about 10 days later, to prevent the spores from falling off the pileus. The best time to pick the reishi is when the spore is brown and on the pileus. The picked reishi mushroom can be heat desiccated until the moisture is only about 8-10%, and then sealed to keep away moisture.

The examples provided below are illustrative of various embodiments of formulating and using reishi extracts. While certain reishi extracts and/or additional ingredients, or combinations of ingredients, may be preferred, no limitation thereto is to be inferred. Rather, the type of reishi formulation desired will dictate which specific components, and amounts thereof, are to be included in addition to the reishi extract. It is to be understood the following examples were conducted on animals, where human formulations may vary with respect to the amount and concentration of any and/or all ingredient(s). These examples are provided to convey a more full understanding of the range of effective reishi formulations included in the present invention, and in no way to act as a limitation thereof.

### EXAMPLES

### Example 1

### Reishi Extract Formulation

Reishi extract formulations were prepared in accordance with the invention. Reishi was grown and produced by using a solid wood log cultivation method. After cultivation, a multi-step extraction process was used for preparation of a reishi fruit and cracked reishi spore composition. The extract was made by a process of concentrating the triterpenes from a reishi mushroom, detaching polysaccharides from cell walls, and concentrating polysaccharides, as described above. The extracts were processed into a powder, and subsequently encapsulated with about 495 mg of reishi fruit extract and 5 mg cracked spores. Accordingly, the capsules were analyzed, and it was determined that the contents amounted to about 99% reishi fruit extract and about 1% cracked reishi spores per capsule. The resulting capsules were analyzed to determine triterpene and polysaccharide content.

### Example 2

### Triterpene Analysis

Capsules made pursuant to Example 1 were obtained and analyzed for triterpene content. For a sample preparation, the capsules were partitioned and a 100 mg sample was accurately weighed. The sample and 5 ml ethyl acetate were placed into a sealed PVC tube. The PVC tube and contained solution were sonicated for 30 minutes in a constant temperature bath. The solution was then centrifuged so that the extracted solution could be pipetted out. Ethyl acetate was then added again to the extracted solution until the sample showed no color. The extracted solution was then dried under vacuum to evaporate the solvent to form a residue. The residue was then dissolved in methanol and diluted to 2 ml, and filtered through a 0.45 micrometer membrane filter prior to HPLC analysis.

For a standard preparation, 1 mg of ganoderic acid A, was added to a 10 ml volumetric flask, and then dissolved in methanol. The solution was then diluted to volume with methanol. The standard solution was then filtered through a 0.45 micrometer membrane filter.

Separately, the standard preparation and sample preparation were injected into the HPLC in 20 microliter amounts. Accordingly, the separate chromatograms were recorded. The total triterpenes in the extract were then calculated by the formula:
Triterpenes %=(A·Cₛ/Aₛ)/(Wₛₐₘₚₗₑ)·(566/500)·100%

Here, A is the total peak area from retention time 8.0 to 38.0 minutes in the sample preparation chromatogram; Aₛ is the peak area of the standard preparation chromatogram; Cₛ is the concentration of the standard preparation (mg/ml); and Wₛₐₘₚₗₑ is the weight (mg) of sample weighed. The total triterpene for three batches of capsules are set forth in Table 1.

TABLE 1
Total Triterpenes

| Capsule Batch No. | Triterpenes per Capsule | % Triterpenes by Weight |
|---|---|---|
| 1 | 31.5 mg | 6.3% |
| 2 | 35.0 mg | 7.0% |
| 3 | 34.5 mg | 6.9% |

The results indicate that reishi extract can be an effective source of triterpenes. More particularly, the results indicate that compositions formulated as in Example 1 with reishi fruit extract and cracked reishi spore extract contain triterpenes between 5 to 10% by total weight.

### Example 3

### Polysaccharide Analysis

Capsules according to Example 1 were obtained and analyzed for polysaccharide content. For a sample preparation, the capsules were partitioned and a 200 mg sample was accurately weighed. The sample was placed in a 100 ml volumetric flask and solubilized with 80 ml water. The solution was sonicated for 20 minutes, and then cooled to room temperature prior to diluting the solution with water to volume. The diluted solution was pipetted at 0.4 ml, which was mixed with 0.8 ml of a 5% phenol solution. The solution was then added to 4 ml of sulfuric acid and shaken prior to allowing the solution to stand for 30 minutes at room temperature.

For a standard preparation, 1.2 mg of glucose was added to a 10 ml volumetric flask and dissolved in water to volume. The solution was pipetted at 0.4 ml, which was mixed with 0.8 ml of a 5% phenol solution. The solution was then added to 4 ml of sulfuric acid and shaken prior to allowing the solution to stand for 30 minutes at room temperature.

Separately, the sample preparation and the standard separation absorbance spectrographs were recorded at 490 nm, with a reference wavelength of 650 nm. Each preparation was measured against the absorbance of a blank preparation of water. The total polysaccharides were calculated by the following formulas:
E=C/(A′₄₉₀−A′₆₅₀)
Polysaccharides(mg)=(E·(A₄₉₀−A₆₅₀)·V·0.91/W)·W_{capsule}

Here, C is the standard preparation concentration of glucose (mg/ml); V is the sample preparation volume (ml); W is the weight of sample preparation (mg); W_{capsule} is the average filling weight per capsule (mg); A₄₉₀ and A₆₅₀ are the absorbance measured for the sample preparation at the indicated wavelength; A′₄₉₀ and A′₆₅₀ are the absorbance measured for the standard preparation at the indicated wavelength. The total polysaccharides for three batches of capsules are set forth in Table 2.

TABLE 2
Total Polysaccharides

| | Polysaccharides | % Polysaccharides |
|---|---|---|
| Capsule Batch No. | per Capsule | by Weight |
| 1 | 69.5 mg | 13.9% |
| 2 | 70.5 mg | 14.1% |
| 3 | 68.0 mg | 13.6% |

The results indicate that reishi extract can be an effective source of polysaccharides. More particularly, the results indicate that compositions formulated as in Example 1 with reishi fruit extract and cracked reishi spore extract contain polysaccharides between 12 to 18% by total weight.

### Example 4

### Ovalbumin-Induced (OVA) Mouse Model

The effect of reishi extract on secretion of IL-5 in Balb/c mice with ovalbumin-induced (OVA) sensitization induced by repeated exposure to OVA was observed. Sensitizing mice to OVA to simulate an allergic model is well known in the art. Each group consisted of 10 mice, where the mice of each group were administered with either a placebo or any of the prescribed amounts of reishi extract formulation for 6 weeks. Different groups of mice were administered reishi extract compositions of Example 1 in the amounts of 0.585 g/kg, 1.170 g/kg, and 2.925 g/kg. The effects of varying the dose of reishi extract on the change in OVA-induced IL-5 secretion were compared to the OVA-induced IL-5 secretion by the mice administered with only a placebo. After 6 weeks the impact of various amounts of reishi compositions in OVA-induced splenocyte IL-5 secretion was recorded, and is set forth in Table 3.

TABLE 3
Splenocyte IL-5 Secretion

| | | Splenocyte IL-5 | % Change from |
|---|---|---|---|
| Amount of Reishi | | Secretion | Placebo |
| Placebo 0 | (g/kg) | ˜1053 (pg/ml) | — |
| 0.0585 | (g/kg) | ˜947 (pg/ml) | ˜−10% |
| 1.170 | (g/kg) | ˜653 (pg/ml) | ˜−38% |
| 2.925 | (g/kg) | ˜716 (pg/ml) | ˜−32% |

The results indicate that dosing mice for 6 weeks with 0.585 g/kg of reishi extract decreased secretion of IL-5 by about 10% compared to the level of IL-5 secretion in the placebo group. Further, the mice dosed with 1.170 g/kg of reishi extract showed about a 38% decrease in IL-5 secretion by the same comparison. Furthermore, the mice dosed with 2.925 g/kg reishi extract decreased IL-5 secretion by about 32% in comparison with the placebo. Accordingly, the reishi extract composition of Example 1 was observed to inhibit IL-5 secretion upon sensitization and exposure to OVA.

It is to be understood that the above-described examples are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and/or preferred embodiments of the invention, it will be apparent to those of ordinary skill in the art that these examples not intended to be limiting in nature.

What is claimed is:

1. A method of inhibiting IL-5 secretion in a subject comprising:
(a) providing a composition containing a therapeutically effective amount of reishi extract, and
(b) administering the composition to the subject.

2. The method of claim 1, wherein the composition includes a cracked reishi spore extract.

3. The method of claim 1, wherein the composition includes a reishi fruit extract.

4. The method of claim 1, wherein the composition includes a reishi fruit extract and cracked reishi spores.

5. The method of claim 4, wherein the composition comprises from about 247.5 to 990 mg of reishi fruit extract and from about 2.5 to 10 mg of cracked reishi spores.

6. The method of claim 4, wherein the composition comprises about 495 mg of reishi fruit extract and about 5 mg of cracked reishi spores.

7. The method of claim 4, wherein the composition is administered in an amount and frequency sufficient to provide a daily dosage of from about 495 to 1980 mg of reishi fruit extract and of from about 5-20 mg of cracked reishi spores.

8. The method of claim 4, wherein the composition is administered in an amount and frequency sufficient to provide a daily dosage of about 990 mg of reishi fruit extract and of about 10 mg of cracked reishi spores.

9. The method of claim 1, wherein the composition is administered in an amount and frequency sufficient to provide a daily dosage of from about 1 to 2 g of reishi extract.

10. The method of claim 1, wherein the reishi extract comprises:
(a) at least one triterpene compound, and
(b) at least one polysaccharide compound.

11. The method of claim 10, wherein the reishi extract comprises from about 5 to 10% triterpene compounds and from about 12 to 18% polysaccharide compounds.

12. The method of claim 4, wherein the reishi extract consists essentially of about 99% reishi fruit and about 1% cracked reishi spore.

13. The method of claim 1, wherein the inhibition of IL-5 secretion treats or prevents an asthmatic condition.

14. The method of claim 1, wherein the inhibition of IL-5 secretion treats or prevents an allergic reaction.

15. A composition comprising a reishi extract, which inhibits IL-5 secretion when administered to a subject in a therapeutically effective amount.

16. The composition of claim 15, wherein the reishi extract comprises:
(a) at least one triterpene compound, and
(b) at least one polysaccharide compound.

17. The composition of claim 16, wherein the composition comprises from about 5 to 10% triterpene compounds and from about 12 to 18% polysaccharide compounds.

18. The composition of claim 15, wherein the composition includes cracked reishi spores.

19. The composition of claim 15, wherein the composition includes a reishi fruit extract.

20. The composition of claim 15, wherein the composition includes a reishi fruit extract and cracked reishi spores.

21. The composition of claim 20, wherein the composition consists essentially of about 99% reishi fruit and about 1% cracked reishi spore.

22. A method for treating or preventing an allergic reaction in a subject comprising:
(a) providing a composition containing a therapeutically effective amount of reishi extract, and
(b) administering the composition to the subject in an amount sufficient to inhibit the secretion of IL-5 in the subject when the subject is exposed to an allergen.

23. The method of claim 22, wherein the allergic reaction is selected from the group consisting of asthma, allergic rhinitis, allergic conjunctivitis and atopic eczema.

24. The method of claim 22, wherein the allergic reaction is a type I reaction.

25. The method of claim 22, wherein the allergic reaction is a type II reaction.

26. The method of claim 22, wherein the allergic reaction is a type III reaction.

27. The method of claim 22, wherein the allergic reaction is a type IV reaction.

28. The method of claim 22, wherein the allergen is ingested.

29. The method of claim 22, wherein the allergen is from dermal contact.

30. The method of claim 22, wherein the allergen is inhaled.

31. The method of claim 22, wherein the allergen is from ingestion of a peanut.

32. The method of claim 22, wherein the allergen is from exposure to a pollen particle.

33. The method of claim 22, wherein the allergen is from exposure to a mold particle.

34. The method of claim 22, wherein the allergen is from exposure to a dust particle.

35. The method of claim 22, wherein the allergen is from exposure to an animal.

36. The method of claim 22, wherein the allergen is an aeroallergen.

37. A method of making a reishi composition comprising:
(a) providing at least a portion of a reishi mushroom,
(b) treating the portion of reishi mushroom with an 65-95% aqueous alcohol solution to obtain a crude triterpene extract,
(c) concentrating the crude triterpene extract to obtain at least one triterpene,
(d) treating the portion of a reishi mushroom with a solvent to obtain a crude polysaccharide extract,
(e) treating the crude polysaccharide extract with ethanol to obtain at least one polysaccharide, and
(f) forming a combined composition by combining at least one triterpene and at least one polysaccharide.

38. The method of claim 37, further comprising:
(a) providing at least a portion of a reishi mushroom including a cell wall, and
(b) detaching at least one polysaccharide from the cell wall to obtain a crude polysaccharide extract.

39. The method of claim 37, wherein treating the crude polysaccharide extract with ethanol precipitates at least one polysaccharide.

40. The method of claim 37, wherein the portion of a reishi mushroom consists essentially of a cracked reishi spore.

41. The method of claim 37, wherein the portion of a reishi mushroom consists essentially of a reishi fruit.

42. The method of claim 37, wherein the portion of a reishi mushroom consists essentially of a cracked reishi spore and a reishi fruit.

43. The method of claim 37, further comprising:
(a) cultivating a reishi mushroom by means of a solid wood log cultivation process, and
(b) partitioning the reishi mushroom into at least one portion.

44. The method of claim 37, wherein the composition consists essentially of about 99% reishi fruit and about 1% cracked reishi spore.

45. The method of claim 37, wherein the composition comprises:
(a) at least one triterpene compound, and
(b) at least one polysaccharide compound

46. The method of claim 45, wherein the composition comprises about 5-10% triterpene compounds and about 12-18% polysaccharide compounds.
